(19) United States
(12) Patent Application Publication
(10) Pub. No.: US 2014/0114387 A1
(43) Pub. Date: Apr. 24, 2014

(54) CHATTER-FREE ACTIVE FIXATION LEAD

(71) Applicant: PACESETTER, INC., Sylmar, CA (US)

(72) Inventors: John W. Poore, South Pasadena, CA (US); Gene A. Bornzin, Simi Valley, CA (US); Zoltan Somogyi, Simi Valley, CA (US); Steven R. Conger, Agua Dulce, CA (US)

(73) Assignee: PACESETTER, INC., Sylmar, CA (US)

(21) Appl. No.: 13/655,125

(22) Filed: Oct. 18, 2012

(51) Int. Cl. A61N 1/05

(52) U.S. Cl. 607/127

(57) ABSTRACT

An implantable therapy lead includes a tubular body, an obturator, and a helical anchor electrode. The obturator is displaceably supported on a distal end of the tubular body between a recessed position and an extended position. When the obturator is in the extended position, the extreme distal tip of the tissue penetrating point of the helical anchor electrode contacts an outer surface of the obturator in a manner that prevents the extreme distal tip from being capable of tissue penetration significant enough to allow the helical anchor electrode to be screwed into the heart tissue. When the obturator is in the recessed position, the extreme distal tip no longer contacts the outer surface of the obturator and the extreme distal tip is positioned relative to the outer surface of the obturator so as to allow the extreme distal tip to penetrate the heart tissue.

### FIELD OF THE INVENTION

The present invention relates to medical apparatus and methods. More specifically, the present invention relates to implantable therapy leads and methods of using such leads.

### BACKGROUND OF THE INVENTION

Implantable therapy leads may be configured for active fixation. A common arrangement for a lead configured for active fixation provides a lead distal end with an active fixation helix that extends from the distal end of the lead when an IS-1 connector pin is rotated at a proximal end of the lead. As the connector pin is rotated clockwise, the sharp helix rotates and extends from the lead distal end to screw into myocardial tissue. Such an active fixation helix arrangement is mechanically complex and expensive to manufacture.

Because the helix can also serve as an electrode for pacing and sensing functions of the lead, such a helix arrangement has the disadvantage that the helix is not fixedly connected to the electrical conductors extending through the lead between the helix and the IS-1 connector pin. The loose connection between the helix and the electrical conductors can cause electrical noise in the sense amplifier of the pacemaker or implantable cardioverter defibrillator (ICD) electrically connected to the IS-1 connector pin. This electrical noise is known as “chatter”.

There is a need in the art for an active fixation lead that is less mechanically complex and expensive to manufacture. There is also a need in the art for an active fixation lead that substantially, if not totally, eliminates chatter associated with the helix electrode circuit.

### SUMMARY

An implantable therapy lead is disclosed herein. In one embodiment, the therapy lead is configured for active fixation to heart tissue. The lead includes a tubular body, an opening, an obturator, and a helical anchor. The tubular body includes a distal end, a proximal end opposite the distal end, and a longitudinal axis extending between the proximal end and distal end. The opening is defined in the distal end generally coaxial with the longitudinal axis. The obturator includes an outer cylindrical surface and is displaceable along the longitudinal axis between a recessed position and an extended position. The obturator is at least substantially located within the distal end proximal the opening when the obturator is in the recessed position, and the obturator extends substantially distal the opening when the obturator is in the extended position. The helical anchor extends from the opening generally coaxial with the longitudinal axis and positionally fixed relative to the distal end. The helical anchor includes a longitudinal center axis and a distal tissue penetrating point. The point is configured such that, when the obturator is in the extended position within the helical anchor, an extreme distal tip of the point and the outer cylindrical surface make surface contact in such a manner that the point is generally prevented from cutting or abraiding the patient's blood vessel wall while the lead is being inserted into the patient. Once the lead tip is located at the location where it is to be screwed into the myocardium, the obturator is allowed to slide back into the lead body toward the proximal end of the lead so that the helix can advance into the tissue.

The lead may also include a connector assembly near the proximal end. The connector assembly includes an electrical contact in electrical communication with the helical anchor. The helical anchor is also configured to act as an electrode in addition to serving as a mechanism for active fixation. In some embodiments, the helical anchor is not electrically active, but simply acts as an anchor. In such a non-electrically active embodiment, the helical anchor may be formed of metal or even of non-electrically conductive materials.

In one embodiment, the obturator is biased towards the recessed position.

In one embodiment, the surface contact is at least partially a result of the tip making generally tangential surface contact with the outer cylindrical surface. In other words, the surface contact is at least partially a result of the tip intersecting the outer cylindrical surface in a generally flush manner.

In one embodiment, the helical anchor includes a wire-like member helically wound into multiple coils. A most distal coil distally terminates in the point and includes a radially inner curved boundary and a radially outer curved boundary opposite the radially inner curved boundary. The point proximally begins on the radially outer curved boundary and distally terminates in the tip at the radially inner curved boundary. The point includes a bevel having a proximal border on the radially outer curved boundary and a distal border in a form of the tip on the radially inner curved boundary. The bevel includes a curved surface or planar surface between the proximal border and the tip. The tip is defined at least in part by an intersection of the radially inner curved boundary and the bevel.

A method of implanting an active fixation implantable therapy lead is also disclosed herein. In one embodiment, the method includes: a) negotiating the lead through a cardiovascular system of a patient with an obturator of the lead in an extended position wherein an extreme distal tip of a tissue penetrating point of a helix anchor electrode contacts an outer surface of the obturator in a manner that prevents the extreme distal tip from being capable of cutting a blood vessel during implanting and preventing the helix anchor electrode from being screwed into tissue; b) allowing the obturator to move to a recessed position wherein the extreme distal tip no longer contacts the outer surface of the obturator and the extreme distal tip is positioned relative to the outer surface of the obturator so as to allow the extreme distal tip to penetrate tissue; and c) with the extreme distal tip and outer surface of the obturator positioned as recited in b), rotating the lead about a longitudinal axis of the lead to cause the helix anchor electrode to screw into the tissue.

Another implantable therapy lead is also disclosed herein. In one embodiment, the therapy lead is configured for active fixation to heart tissue. The lead includes a tubular body, an obturator, and a helical anchor electrode. The tubular body includes a distal end, a proximal end opposite the distal end, and a longitudinal axis extending between the proximal end and distal end. The obturator is displaceably supported on the distal end between a recessed position and an extended position. The helical anchor electrode is fixedly supported on the distal end and includes a tissue penetrating point including an extreme distal tip. When the obturator is in the extended position, the extreme distal tip of the tissue penetrating point of the helical anchor electrode contacts an outer surface of the obturator in a manner that prevents the extreme distal tip from being capable of tissue penetration significant enough to allow the helical anchor electrode to be screwed into the heart tissue. When the obturator is in the recessed position, the extreme distal tip no longer contacts the outer surface of the obturator and the extreme distal tip is positioned relative to the outer surface of the obturator so as to allow the extreme distal tip to penetrate the heart tissue.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a side plan view of an embodiment of the lead.

FIG. 2 is a longitudinal cross-section of the electrode assembly where an obturator is in a recessed state.

FIG. 3 is the same view as FIG. 2, except the obturator is shown in an extended state.

FIG. 4 is an elevation view of the lead distal end as viewed in the direction indicated by line 4-4 in FIG. 3.

FIG. 5 is the same view as FIG. 4, except showing only the helically coiled anchor electrode for clarity purposes.

### DETAILED DESCRIPTION

### a) Overview

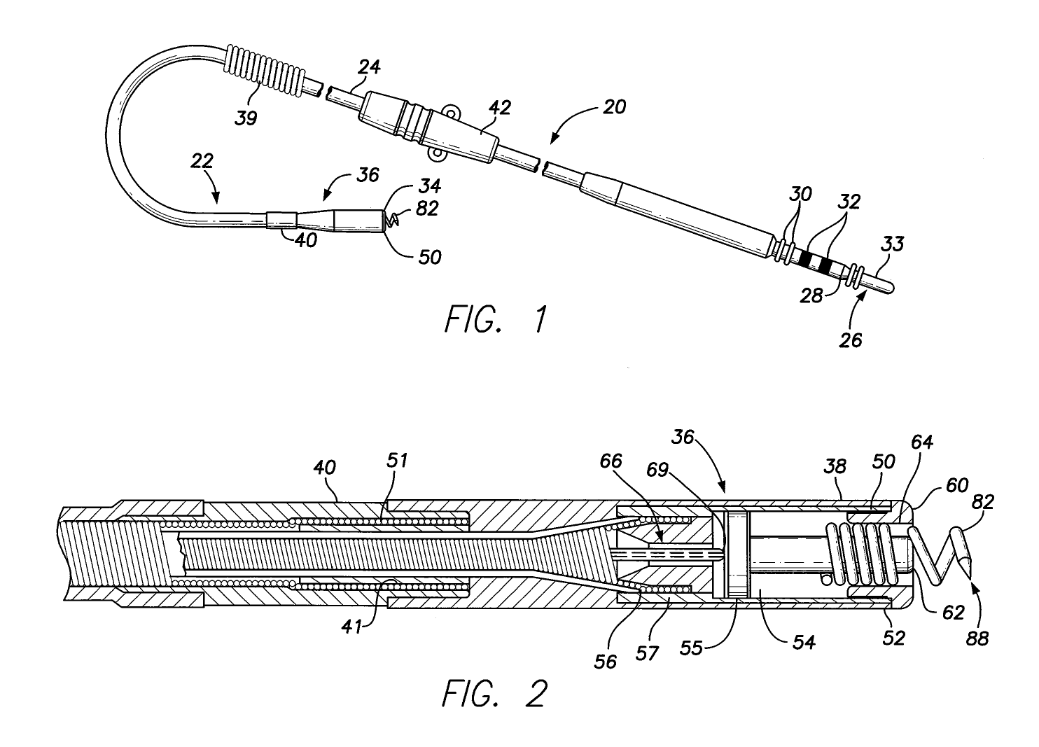

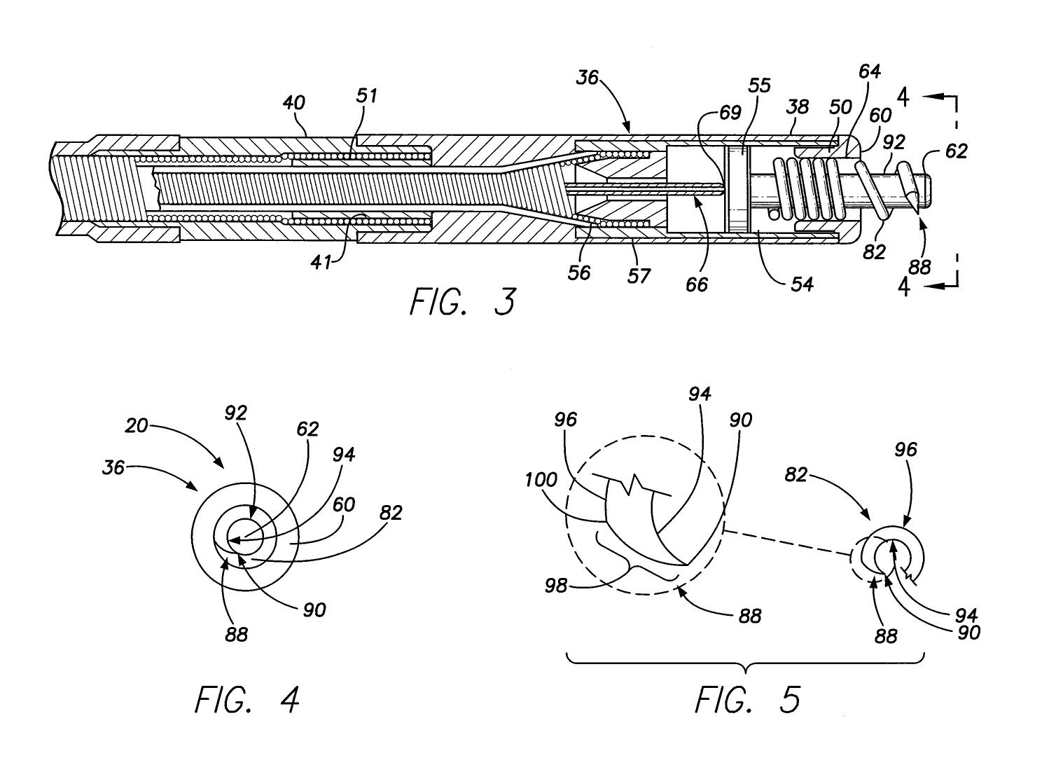

An implantable therapy lead 20 (e.g., a CRT lead, etc.) and a method of using such a lead are disclosed herein. In one embodiment, the therapy lead 20 is configured for active fixation to heart tissue. The lead 20 includes a tubular body 22, an opening of a bore 64, an obturator 62, and an active fixation helical anchor electrode 82. The tubular body includes a distal end 34, a proximal end 26 opposite the distal end, and a longitudinal axis extending between the proximal end and distal end. The opening of the bore 64 is defined in the distal end 34 generally coaxial with the longitudinal axis. The obturator 62 includes an outer cylindrical surface 92 and is displaceable along the longitudinal axis between a recessed position (see FIG. 2) and an extended position (see FIG. 3). The obturator 62 is at least substantially located within the distal end 34 proximal the opening of the bore 64 when the obturator is in the recessed position, and the obturator 62 extends substantially distal the opening of the bore 64 when the obturator 62 is in the extended position.

The helical anchor 82 extends from the opening of the bore 64 generally coaxial with the longitudinal axis and positionally fixed relative to the distal end 34. The helical anchor 82 includes a longitudinal center axis and a distal tissue penetrating point 88. The point 88 is configured such that, when the obturator 62 is in the extended position within the helical anchor 82, an extreme distal tip 90 of the point 88 and the outer cylindrical surface 92 make surface contact in such a manner that the point 88 is generally prevented from biting into the blood vessels or heart tissue when the helical anchor 82 is moved or rotated against tissue.

At least in part because the active fixation helix anchor electrode 82 is permanently fixed to the both the structure of the distal region of the lead body and the electrical conductor extending between the helix electrode 82 and the pin contact 33 of the connector assembly 28, the helix electrode configuration disclosed herein is both electrically and mechanically stable. As a result, the helix electrode configuration eliminates eliminating (or at least substantially reduces) the associated electrical noise (i.e., chatter) and also reduces the associated manufacturing complexity and costs.

### b) Device

To begin a detailed discussion of the lead 20, reference is made to FIG. 1, which is a side plan view of an embodiment of the lead 20. As can be understood from FIG. 1, the lead 20 is designed for intravenous insertion and contact with the endocardium, and as such, may be conventionally referred to as an endocardial lead. As indicated in FIGS. 1 and 2, the lead 20 is provided with an elongated lead body 22, which includes coiled or helically wound electrical conductors 51 and 56 covered with an insulation sheath 24. The insulation sheath is preferably fabricated of silicone rubber, polyurethane, silicone rubber—polyurethane—copolymer (SPC), or other suitable plastic. At a proximal end 26 of the lead 20 is a connector assembly 28, which is provided with sealing rings 30 and carries at least one or more electrical connectors in the form of ring contacts 32 and a pin contact 33. A helical active fixation anchor 82 distally extends from a distal end 34 of the lead 20. The anchor 82 may also be configured to act as an electrode in addition to providing active fixation.

The connector assembly 28 is constructed using known techniques and is preferably fabricated of silicone rubber, polyurethane, SPC, or other suitable plastic. Electrical contacts 32, 33 are preferably fabricated of stainless steel or other suitable electrically conductive material. The lead 20 is constructed to include a hollow interior extending from the proximal end 26 to a distal end 34. The hollow interior allows for the introduction of a stylet, guidewire or other device during implant, which is beneficial in allowing the surgeon to guide the otherwise flexible lead 20 from the point of venous insertion to the myocardium.

As shown in FIG. 1, at the distal end 34 of the pacing lead 20 is an electrode assembly 36, which is discussed in more detail below. A fixation sleeve 42, slidably mounted around lead body 22, serves to stabilize the pacing lead 20 at the site of venous insertion. Where the lead 20 is equipped for defibrillation, a shock coil 39 will be supported on the lead body 22 proximal the electrode assembly 36 and distal the fixation sleeve 42. The shock coil 39 is electrically coupled to one of the ring contacts 32 of the connector assembly 28 via electrical conductors extending through the lead body 22 in the form of wires, cables or other electrical conductors that are linear or helically coiled in configuration.

The construction of the electrode assembly 36 of FIG. 1 is shown in greater detail in FIGS. 2 and 3, which are longitudinal cross-sections of the electrode assembly where an obturator 62 is in a recessed state and an extended state, respectively. As illustrated in FIGS. 2 and 3, the electrode assembly 36 at the distal end of the lead 20 includes a conductive electrode 50 and located about the distal end 34 of the electrode assembly 36 is an insulating sheath 38 that extends from the distal end of lead body 22 to an annular ring electrode 40.

Lead conductor 51 is crimped to crimp tube 41, which is in electrical contact with ring electrode 40, thereby establishing an electrical connection between conductor 51 and electrode 40. The conductor 51 is in electrical communication with one of the ring contacts 32 of the connector assembly 28.

The conductive electrode 50 is preferably a unitary construction including at its proximal end a cylindrical portion 52 being secured by means of a press fit in an axial bore 54 that is defined by conductive annular sleeve 57. The helical coil conductor 56 extends through the lead body 22 of FIG. 1 from the pin contact 33 of the connector assembly 28. A distal region of the helical coil conductor 56 is electrically coupled, typically by way of crimping and/or welding, to the annular sleeve 57. The sleeve 57 extends substantially to the distal end 34 where, as just described, electrical contact is made with electrode 50 via the aforementioned press fit and/or crimping and/or welding.

As illustrated in FIGS. 2 and 3, the helical coil conductor 56 defines the walls of the hollow interior of the lead 20, which accepts a delivery tool such as, for example, stylet 66 during insertion. Stylet 66 may be coaxial with a base 55, the stylet 66 and base 55 being extendable together to displace the obturator 62 as discussed below. The base 55 is located in bore 54. The base 55 is longitudinally slidable within bore 54 under the action of stylet 66.

The electrode distal tip 60 is depicted as including an internal bore 64 defined by the inner annular surface of conductive electrode 50. The electrode materials for the electrode distal tip 60 are preferably a base metallic material, optimally a platinum-iridium alloy or similarly conductive biocompatible material. In one embodiment, the platinum-iridium alloy has a composition of about 90% platinum and 10% iridium by weight.

As indicated in FIGS. 2 and 3, the electrode distal tip 60 also includes an active fixation helix anchor 82 which is mounted on, and fixedly attached to, inner circumferential surface 64 of the distal electrode 50 via, for example, mechanical press fit, crimping, and/or laser welding. In some embodiments, the helix anchor 82 not only serves as an active fixation anchor 82, but also is configured to serve as an electrode. Thus, in such an embodiment, the helix anchor electrode 82 is both an active fixation anchor and an electrode. In such an embodiment, the fixation of the helix electrode 82 to the distal electrode 50 is such that the helix electrode 82 is positionally fixed relative to the distal electrode 50 and there is good electrical communication between the two, the electrical communication being such that there is essentially no electrical noise (i.e., chatter) associated with the fixed mechanical and electrical connection between the distal electrode 50 and the helix anchor electrode 82.

As shown in FIGS. 1-3, the helix electrode 82 is centrally disposed with respect to the distal tip 60 and is permanently fixed in a distally extended relationship relative to the distal tip 60 such that a number of coils and the distal tissue penetrating point 88 of the helix electrode 82 are always distal the distal tip 60. In addition to be permanently fixed to the distal structure of the lead body, the helix electrode 82 is also permanently fixed to the electrical conductor extending from the helix electrode 82 to the contact pin 33. The helical anchor electrode 82 includes a wire-like member helically wound into multiple coils, a most distal coil distally terminating in the point 88.

For a detailed discussion of the helix anchor electrode 82 in the vicinity of the point 88, reference is now made to FIGS. 4 and 5, wherein FIG. 4 is an elevation view of the lead distal end as viewed in the direction indicated by line 4-4 in FIG. 3, and FIG. 5 is the same view as FIG. 4, except showing only the helically coiled anchor electrode 82 for clarity purposes. As illustrated in FIGS. 4 and 5, the most distal coil of the helix anchor electrode 82 includes a radially inner curved boundary 94 and a radially outer curved boundary 96 opposite the radially inner curved boundary 94. The most distal coil of the helix anchor electrode 82 distally terminates in the sharp tissue penetrating point 88.

As best understood from the enlarged view of the point 88 depicted in FIG. 5, the point 88 proximally begins on the radially outer curved boundary 96 and distally terminates in a sharp tip 90 at the radially inner curved boundary 94, the sharp tip 90 forming the extreme distal termination of the tissue penetrating point 88. The point 88 can be seen to include a grind, taper or bevel surface 98 that have a proximal border 100 on the radially outer curved boundary 96 and a distal border in a form of the sharp tip 90 on the radially inner curved boundary 94. In one embodiment, the bevel 98 may have a curved surface between the proximal border 100 and the tip 90. In other embodiments, the bevel 98 may have a straight, flat or planar surface between the proximal border and the tip. The tip 90 is defined at least in part by an intersection of the radially inner curved boundary 94 and the bevel 98.

As can be understood from FIGS. 2 and 3, disposed within the internal bore 64 is an obturator 62 that is cylindrically shaped and includes a proximal end that is coupled to a distal face of the base 55 such that the obturator 62 extends distally from the base 55 through the center of the helically coiled electrode 82 that forms the active fixation electrode anchor 82 at the lead distal end 34. The obturator 62 is axially movable relative to the helically coiled electrode 82 and the rest of the distal region of the lead 20. Specifically, the obturator 62 can be caused to displace between a recessed position wherein the obturator 62 is at least substantially within the confines of the distal end 34 (see FIG. 2) and an extended position wherein the obturator 62 is substantially distal the distal end 34 and extends through the coils of the helix anchor electrode 82 generally coaxial with the longitudinal axis of the helix anchor electrode 82 (see FIG. 3). The obturator proximal end is fixedly attached to the distal face of the base 55, typically by laser welding, adhesive, or mechanical methods, such as, a fastener or crimping. Alternatively, the obturator 62 may simply be an extension of the base 55.

The obturator 62 may be formed of an electrically non-conductive, biocompatible material. In one embodiment, the obturator 62 may be configured to contain and deliver over time a therapeutic agent. For example, in one embodiment, the obturator 62 may be at least partially formed of or support a mixture of copolymeric Lactic/Glycolic acid (PLA/GLA), polylactic acid, polyglycolic acid, polyamino acid, or polyorthoester and a desirable therapeutic, up to 50% by weight, such as dexamethasone sodium phosphate for the minimization of inflammation resultant from foreign body reactions to the surrounding tissue. The obturator releases the desired therapy (e.g., steroid) over time to counter the commonly known undesirable side effects of the implant, i.e., inflammation. Because the obturator 62 with its therapeutic are at the center of the helical electrode 82, the therapeutic can be delivered to the myocardium, very close to the site of implantation of the helical electrode 82.

As can be understood from a comparison of FIGS. 2 and 3, the obturator 62 is extendable from within the internal bore 64 when the stylet 66 is urged against base 55. Screwing the anchor 82 into tissue brings the distal end of the obturator 62 into contact with the tissue and, as the anchor 82 is increasingly screwed into the tissue, the tissue pushes proximally against the distal end of the obturator 62, thereby causing the obturator 62 to increasingly recess back into the axial bore 54.

In an alternative embodiment, a helical spring (not shown) may be positioned in the bore 54 to act between the distal face of the base 55 and the proximal edge of the conductive electrode 50 located in the bore 54, thereby biasing the obturator 62 proximally to recess the obturator 62 within the confines of the distal end of the lead unless distally displaced by the stylet 66 urging the base 55 and the obturator 62 distally. Thus, to place anchor 82 in condition to be screwed into tissue, the stylet only needs to cease pushing distally on the base 55, thereby allowing the obturator 62 to recess to expose the helix tip 88 such that the helix tip will be able to bite into tissue.

As can be understood from FIGS. 3 and 4, when the obturator 62 is fully distally extended as indicated in FIG. 3, the outer cylindrical surface 92 of the obturator 62 fills the cylindrical void defined by the inner cylindrical boarder 94 of the helically coiled electrode 82 such that the two cylindrical boundaries generally intersect along the lengths of the obturator and helically coiled electrode. As illustrated in FIGS. 4 and 5, the tissue penetrating tip 88 of the anchor 82 is configured such that it terminates to be generally flush against the outer cylindrical surface 92 of the obturator 62. More specifically, distal tissue penetrating point 88 is configured such that, when the obturator 62 is in the extended position (see FIG. 3) within the helical anchor 82, an extreme distal tip 90 of the point 88 and the outer cylindrical surface 92 of the obturator 62 make surface contact in such a manner that the point 88 is generally prevented from cutting or snagging the blood vessel during inserting or biting into heart tissue when the helical anchor 82 is rotated about the longitudinal center axis of the helical anchor 82. As can be understood from FIG. 4, the ability of the point 88 to not cut snag or bite into tissue when the obturator is in the extended position through the coils of the helix anchor electrode and the helix anchor electrode is being inserted through blood vessels or rotated against the heart tissue is at lest in part a result of the tip 90 of the point 88 making generally tangential surface contact with the outer cylindrical surface 92 of the obturator 62. In other words, this advantageous surface contact is at least partially a result of the tip 90 of the point 88 intersecting the outer cylindrical surface 92 of the obturator 62 in a generally flush manner. Rather than pointing away from the obturator outer surface 92 and towards the blood vessel or heart tissue, the sharp tip 90 of the point 90 can be seen to be against the outer surface 92 of the obturator 62, thereby keeping the point 88 from biting into and engaging tissue.

### c) Method of Use

For a discussion of a method of employing the lead disclosed herein, reference is made to FIGS. 1-5. As can be understood from FIGS. 1-5, to prevent the sharp helix point 88 from damaging tissue as the lead 20 is advanced through the vasculature and into the heart and, in the case of a ventricular lead, past the tricuspid valve to the ventricular apex, a round tipped obturator 62 fills the helix anchor electrode 82 as long as a stylet 66 extending through the lead body 22 pushes the lead 20 to its final location. When the stylet 66 is pulled back, the obturator 62 freely retracts as the helix anchor electrode 82 is screwed into myocardium. The helix anchor electrode 82 is screwed into the myocardium by rotating the lead body 22 clockwise. Unlike current lead designs, the IS-1 pin 33 does not need to rotate within the connector 28.

As can be understood from FIGS. 3 and 4, in negotiating the lead through the vasculature and heart chambers to the implantation site, the stylet 66 acts against the base 55 so as to cause the obturator 62 to extend out the lead distal end 34 to fill the center volume of the helix anchor electrode 82 and cause the tip 90 of the helix point 88 to be generally flush with the obturator outer surface 92. With the tip 90 so arranged relative to the obturator outer surface 92, it is impossible for the helix point 88 to bite into, pierce or otherwise engage the vasculature or myocardium in any significant manner that would allow the helix anchor electrode 82 begin to cut, scrape or screw into the vasculature or myocardium. Thus, during the time that the lead 20 is being positioned and advanced with the stylet 66, the obturator 88 is securely in place protecting the patient's tissue from the sharp tip 90 of the helix point 88.

As represented in FIG. 2, the obturator 62 is recessed so as to allow the helix 82 to be capable of being screwed into myocardium. Specifically, with the stylet 66 not pushing the obturator 62 into the extended position, tissue contacting the distal end of the obturator 62 in the course of screwing the helix 82 into the tissue causes the obturator 62 to recess within the bore 54 as the obturator 62 is free to slide into the recessed position. As the obturator no longer extends through the center of the helix 82, the sharp tip 90 of the helix point 88 is no longer blocked from engaging myocardium by the interface of the sharp tip 90 with the obturator outer surface 92. Thus, with the sharp tip 90 of the helix point 90 exposed sufficiently to allow the tip 90 to bite into, pierce or otherwise engage the myocardium, the helix 82 is screwed into the myocardium by rotating the entire lead 20 about the longitudinal axis of the lead while the exposed and available sharp tip 90 contacts the myocardium.

As indicated in FIG. 2, when the obturator 62 is in the recessed position, the obturator distal face is generally flush with the face of the distal end of the lead body or end electrode 60. As a result, the obturator distal face in combination with the lead body distal end face provide a substantial area of contact with the myocardium for mechanical stability against the myocardium.

If helix electrode relocation is needed after the helix is screwed into the myocardium, the stylet can be reinserted, the helix unscrewed via counter-clockwise rotation of the entire lead body, and the obturator will slide back into the helix by gentle pressure on the stylet. The helix electrode can then be relocated.

The mechanical characteristics of the helix tip and obturator tip design allow electrical mapping during placement of the electrode. At the candidate site, the helix can be pressed against the myocardium with the obturator released. Pacing and sensing thresholds can then be assessed and if adequate, the helix can be screwed into the myocardium. If thresholds are not adequate, the obturator can be re-extended and the lead tip moved to another site.

An additional advantage with respect to torque transfer is provided by the lead embodiment disclosed herein. For example, commonly known leads often require about a five to one turn ratio between the lead connector and the helix, which means that 10-15 turns are required at the connector to fix the helix. With the lead embodiment disclosed herein, because the helix is fixedly coupled to the lead body, the entire lead body structure can be used to transmit torque, not just the inner conductor coil and, as a result, fewer rotations are need to fix the helix in tissue.

Although the present invention has been described with reference to preferred embodiments, persons skilled in the art will recognize that changes may be made in form and detail without departing from the spirit and scope of the invention.

What is claimed is:

1. An implantable therapy lead configured for negotiating through vasculature of a patient and active fixation to heart tissue, the lead comprising:
a tubular body including a distal end, a proximal end opposite the distal end, and a longitudinal axis extending between the proximal end and distal end;
an opening defined in the distal end generally coaxial with the longitudinal axis;
an obturator including an outer cylindrical surface and displaceable along the longitudinal axis between a recessed position and an extended position, wherein the obturator is at least substantially located within the distal end proximal the opening when the obturator is in the recessed position and the obturator extends substantially distal the opening when the obturator is in the extended position; and
a helical anchor extending from the opening generally coaxial with the longitudinal axis and positionally fixed relative to the distal end, the helical anchor including a longitudinal center axis and a distal tissue penetrating point.

2. The lead of claim 1, further comprising a connector assembly near the proximal end and including an electrical contact in electrical communication with the helical anchor, the helical anchor configured to act as an electrode.

3. The lead of claim 1, wherein the obturator is biased towards the recessed position.

4. The lead of claim 1, wherein the point is configured such that, when the obturator is in the extended position within the helical anchor, an extreme distal tip of the point and the outer cylindrical surface make surface contact in such a manner that the point is generally prevented from cutting the vasculature during insertion into the patient or biting into the heart tissue when the helical anchor is rotated about the longitudinal center axis of the helical anchor.

5. The lead of claim 4, wherein the surface contact is at least partially a result of the tip making generally tangential surface contact with the outer cylindrical surface.

6. The lead of claim 4, wherein the surface contact is at least partially a result of the tip intersecting the outer cylindrical surface in a generally flush manner.

7. The lead of claim 4, wherein the helical anchor includes a wire-like member helically wound into multiple coils, a most distal coil distally terminating in the point and including a radially inner curved boundary and a radially outer curved boundary opposite the radially inner curved boundary.

8. The lead of claim 4, wherein the point proximally begins on the radially outer curved boundary and distally terminates in the tip at the radially inner curved boundary.

9. The lead of claim 4, wherein the point includes a bevel comprising:
a proximal border on the radially outer curved boundary; and a distal border in a form of the tip on the radially inner curved boundary.

10. The lead of claim 9, wherein the bevel comprises a curved surface or a planar surface between the proximal border and the tip.

11. The lead of claim 9, wherein the tip is defined at least in part by an intersection of the radially inner curved boundary and the bevel.

12. A method of implanting an active fixation implantable therapy lead, the method comprising:
a) negotiating the lead through a cardiovascular system of a patient with an obturator of the lead in an extended position wherein an extreme distal tip of a tissue penetrating point of a helix anchor electrode contacts an outer surface of the obturator in a manner that prevents the extreme distal tip from being capable of cutting the vasculature of the patient during insertion or tissue penetration significant enough to allow the helix anchor electrode to be screwed into the tissue;
b) allowing the obturator to move to a recessed position wherein the extreme distal tip no longer contacts the outer surface of the obturator and the extreme distal tip is positioned relative to the outer surface of the obturator so as to allow the extreme distal tip to penetrate tissue; and
c) with the extreme distal tip and outer surface of the obturator positioned as recited in b), rotating the lead about a longitudinal axis of the lead to cause the helix anchor electrode to screw into the tissue.

13. The method of claim 12, wherein, in being in the extended position, the obturator extends through a center of the helix anchor electrode.

14. The method of claim 12, wherein the helix anchor electrode is fixed relative to a body of the lead so as to permanently extend from a distal end of the lead body.

15. The method of claim 12, further comprising extending a delivery tool through the lead to cause the obturator to move into the extended position.

16. The method of claim 15, wherein the delivery tool includes a stylet.

17. The method of claim 12, wherein, in allowing the obturator to move to the recessed position, the obturator is allowed to bias to the recessed position.

18. The method of claim 12, wherein, in the extreme distal tip of the tissue penetrating point of the helix anchor electrode contacting the outer surface of the obturator in a manner that prevents the extreme distal tip from being capable of damaging the vasculature during insertion into the patient or tissue penetration significant enough to allow the helix anchor electrode to be screwed into the tissue, the contacting is at least partially a result of the extreme distal tip making generally tangential surface contact with the outer surface.

19. The lead of claim 12, wherein, in the extreme distal tip of the tissue penetrating point of the helix anchor electrode contacting the outer surface of the obturator in a manner that prevents the extreme distal tip from being capable of tissue penetration significant enough to allow the helix anchor electrode to damage the vasculature during insertion into the patient or to be screwed into the tissue, the contacting is at least partially a result of the extreme distal tip intersecting the outer surface in a generally flush manner.

20. An implantable therapy lead configured for negotiating through vasculature of a patient and active fixation to heart tissue, the lead comprising:
a tubular body including a distal end, a proximal end opposite the distal end, and a longitudinal axis extending between the proximal end and distal end;
an obturator displaceably supported on the distal end between a recessed position and an extended position; and
a helical anchor electrode fixedly supported on the distal end and including a tissue penetrating point including an extreme distal tip;
wherein, when the obturator is in the extended position, the extreme distal tip of the tissue penetrating point of the helical anchor electrode contacts an outer surface of the obturator in a manner that prevents the extreme distal tip from being capable of damaging the vasculature during insertion into the patient or tissue penetration significant enough to allow the helical anchor electrode to be screwed into the heart tissue; and
wherein, when the obturator is in the recessed position, the extreme distal tip no longer contacts the outer surface of the obturator and the extreme distal tip is positioned relative to the outer surface of the obturator so as to allow the extreme distal tip to penetrate the heart tissue.

21. The lead of claim 20, wherein, in being in the extended position, the obturator extends through a center of the helical anchor electrode.
